(19) United States
(12) Patent Application Publication
(10) Pub. No.: US 2004/0052827 A1
(43) Pub. Date: Mar. 18, 2004

(54) Cosmetic pencil

(76) Inventors: Wolfgang Winkler, Lauf (DE); Claudia Zarling, Nuremberg (DE); Simona Lebok, Nuremberg (DE)

Correspondence Address: Norris McLaughlin & Marcus, 30th Floor, 220 East 42nd Street, New York, NY, 10017, US

(21) Appl. No.: 10/381,382

(22) PCT Filed: Sep. 27, 2001
(86) PCT No.: PCT/EP01/11215

(30) Foreign Application Priority Data
Sep. 29, 2000 (DE) 200168266

(51) Int. Cl. A61K 6/00

(52) U.S. Cl. 424/401

(57) ABSTRACT

The invention relates to a cosmetic preparation in the form of a lead, especially for lipsticks, lip salve sticks, concealer, blusher, eyeshadow pencils, lipliner pencils, eyeliner pencils, eyebrow pencils, sun protection sticks, anti-acne sticks and the like. The inventive preparation can be produced by extrusion, the leads obtained thereby then being glued. The preparation is free of solid triglycerides, has a melting range of between 30 and 60° C. and contains certain esters combined with silicon derivatives such as silicon wax, silicon polymers and/or silicon elastomers. Optionally, the preparation can contain pigments, extenders, anti-sedimentation agents and standard oil components and wax.

### DESCRIPTION

The invention concerns a cosmetic preparation as set forth in claim 1, preferably in the form of pencils. In particular this invention concerns lipsticks, lip salve sticks, concealer sticks, blusher, eyeshadow pencils, lipliner pencils, eyeliner pencils, eyebrow pencils, sun protection sticks, anti-acne sticks and comparable products, the leads of which were produced in known manner by the extrusion of a suitable material and provided with a casing of wood, a wood substitute or a plastic material. In principle it is also possible for the lead material to be introduced in known manner in a hot condition into a suitable casing of an injectable material and allowed to become cold therein. The suitable processes for sheathing shaped lead materials are basically state of the art which is known from the production of pencils and color crayons and which was already applied in 1927 to the production of extruded cosmetic pencils.

It is basically also known for the leads to be produced and subjected to further processing in different diameters—depending on the respective desired purpose of use—as a liner pencil for drawing lines or as a pencil for application to a surface—.

The consumer wants textures which afford good application properties. Cosmetic pencils of the kind set forth in the opening part of this specification are to be such that they can be applied softly and uniformly, they draw with sharp contour lines and they remain at the location of application for as long as possible. They should therefore not be caused to come off due to skin moisture, perspiration or sebum and as far as possible they should also not be transferred to other media such as glass or textiles.

In the case of extruded leads it is known state of the art to process a mixture of triglycerides, waxes and pigments—the pigment content is preferably of an order of magnitude of 40-50% by weight—to form leads. Leads produced in that way are admittedly distinguished by a copious application of color but they are relatively hard and are therefore preferably suitable for eyebrow pencils as well as eyeliner and lipliner pencils. Such cosmetic materials are rather unsuitable, because of their hardness and the mostly blunt application thereof, for the production of eyeshadow pencils. A reduction in the pigment content and setting the materials to be softer generally results in problems in terms of covering power and reduced temperature stability. True to the ‘principle of the lowest force’ (Le Chatelier, 1888), cosmetic materials which contain triglycerides, due to the high pressure involved in the extrusion operation, generally involve phase transformation phenomena in crystal modifications of relatively small volumes, which after the extrusion procedure often results in materials which are rubber-like soft and which become harder materials again only after relatively long storage—this is the known ‘post-hardening’ of the leads, with often unwanted effluorescence phenomena on the lead surface.

Another process for the production of leads is casting mixtures, which are molten at elevated temperature, of oils, fats, waxes and pigments in casting molds which generally comprise metal, and further processing of the castings after they have cooled or direct casting of the molten materials in suitable sharpenable casings or sheaths or in suitable rotational mechanisms. In the casting processes the amount of pigments used is limited as pigment concentrations of over 25-30% by weight in most cases increase the viscosity of the fluid materials to such a very great extent that casting is no longer possible. In principle it is also possible for the materials which are fluid at elevated temperature to be pressed under pressure into molds or suitable casings or sheaths—in this case, by virtue of the above-mentioned ‘Le Chatelier principle’ shrinkage procedures also occur, which generally result in the known deep funnel configurations at the rear end of the casting when it has cooled. In order to achieve a low viscosity in the molten condition, oils also have to be added to those cosmetic materials, and such oils are later disadvantageous in terms of the durability of those materials. It was possible to achieve a certain remedy by adding to those cosmetic materials volatile constituents, preferably straight short-chain silicone oils, cyclic silicones, suitable isoparaffins or mixtures thereof, which markedly improved subsequent durability of the cosmetic materials produced in that way. A disadvantage in that respect however is that cosmetic pencils produced in that way are introduced into hermetically sealing rotational mechanisms or into suitable sheaths or casings of plastic material and later had to be closed by means of sealingly closing caps in order to prevent the materials from drying out, due to evaporation of the volatile constituents. Casting such materials in wooden casings which were untreated on the inside is not possible for the reason that on the one hand the air included in the wood expands upon coming into contact with the hot material and imparts a spongy-porous structure to the materials while still fluid, and as on the other hand the volatile constituents diffuse through the wood and then cause shrinkage of the leads which are cast therein. Processes which provide for sealing off the insides of wooden casings have long been known but nonetheless the cosmetic pencils produced in that way were unable to gain success in the market.

For the production of cosmetic materials which are to be applied to the skin and which are to adhere thereto, it has been proposed on many occasions that silicone compounds should be used. Thus A Domsch in his book ‘Die kosmetischen Präparate’ [‘The Cosmetic Preparations’ ], volume III, Lipidhaltige und emulgierte Formulierungen [‘Lipid-bearing and emulsified formulations’], 4th edition (1994), Verlag für chemische Industrie, Augsburg, describes compositions for make-up pencils which can be formed from wax, volatile silicones, pigment and fatty acid ethers and esters. The amount of pigment in those pencils it will be noted is only between 20 and 20%.

JP-A 07 233 025 proposes adding a polyether-modified silicone for improving the capacity for adhesion of a cosmetic composition to the skin.

The addition of silicone-bearing compounds is also provided in accordance with EP-A 0 850 643 and EP-A 0 850 644 for the production of cosmetic preparations, in particular for soft pastes. The use of the most widely varying esters in the form of oils, fats or also synthetic esters is also described therein. Thus for example cosmetic preparations are described, which contain ester-bearing oils. It will be noted however that the materials obtained in accordance with those two publications, upon extrusion, form soft pastes which are thus not suitable for the production of cosmetic pencils.

JP-A 2 229 106 discloses solid cosmetic materials which, for improving the properties thereof, contain a polyvalent metal salt of a dialkyl phosphate besides one or more lipophilic esters, silicone oil, a cosmetic oil and pigment. The make-up which is to be produced therewith is distinguished in that it can be easily applied and has a high level of covering power.

In addition EP-A 0 660 701 and EP-A 0 685 226 disclose cosmetic sun protection means which contain special UV light-filtering compounds and in addition also an oil which is selected from polyvalent esters. Those compositions are formulated in the form of lotions, gels, emulsions, creams, milk and the like.

Now, taking those known compositions as the basic starting point, the object of the invention was to develop a cosmetic material which is suitable for the production of extruded leads and which, in particular for eyeshadow pencils and blusher, permits application over a surface, and which in addition can also be used for liner pencils—that is to say cosmetic pencils which exhibit the same properties of use as the products which are to be obtained by casting suitable materials at elevated temperature. Leads of different diameters, which are produced in that manner, are preferably glued into wood or wood substitute, after the extrusion operation, and are subjected to further processing to form finished pencils. They are intended to be distinguished by a creamy soft application behaviour, improved durability and a reduced level of transfer on to other media, good storage capability without a change in the properties of use thereof, and good temperature stability. Another aim was to provide that the development of such cosmetic materials did not involve using volatile constituents.

The object of the invention is attained by the cosmetic composition defined in claim 1 and the use defined in claims 15 and 16.

Surprisingly it was found that, by the combination of the specially selected ester with the special silicone wax and by foregoing the use of triglycerides with a melting range of between 30 and 60° C., it is possible to produce materials which can be formed by extrusion to give leads which enjoy excellent cosmetic and mechanical properties.

The object of the invention is thus attained by waiving the use of solid triglycerides, in particular triglycerides with a melting range of between 30 and 60° C. They were replaced by specific esters of straight-chain or branched carboxylic acids with straight-chain or branched alcohols with a chain length of between C₁ and C₂₄, preferably by esters of citric acid with medium chain lengths in respect of the alkyl chains, for example tri-(cetyl-/stearyl)-citrate and/or methylglucose distearate and/or palmitoyl-/stearoyl monoglyceride or mixtures thereof. Jojoba oil or isostearyl isostearate have also proven to be suitable. Particularly good results were achieved if said esters were combined with silicone waxes such as for example stearyl dimethicone and polymer silixoysilicates of the general formula [(CH₃)₂SiO_{1/2}]ₓ [SiO₂]_{y} or silicone elastomers of the type dimethicone/vinyidimethicone crosspolymer or mixtures of said silicone derivatives. Polymer siloxysilicates which are suitable for the present invention are described for example in the ‘International Cosmetic Ingredient Dictionary and Handbook’, 7th edition, 1997, volume 2, and are commercially available. The siloxysilicates correspond to the above-specified formula, wherein preferably those siloxysilicates are selected, in which in the formula x and y assume such values that in each case the desired properties are achieved, as is known to the man skilled in the art. In this case the ratio of the ester components to silicone components is in a range of between 1:5 and 2:1. A preferred range is between 1:4 and 1:1.

In a preferred embodiment the ester used is an ester of citric acid with cetyl and/or stearoyl alcohol, a monoglyceride of palmitic and/or stearic acid or an ester which is derived from methylglucose and stearic acid. In another preferred embodiment the ester used is a saccharose ester, in particular a saccharose ester of fatty acids with a medium chain length such as sucrose laurate, sucrose myristate, sucrose palmitate, sucrose stearate, sucrose tetrastearate triacetate or a mixture thereof, preferably the higher-melting sucrose tetrastearate triacetate, optionally mixed with another of the above-described esters.

It was found that, to produce the desired cosmetic materials involving creamy application and long durability on the skin, the above-mentioned esters and silicone derivatives can be combined without any problem with usual oil components such as for example caprylic/capric triglycerides, octyldodecanol, butylstearate and with usual wax components such as for example beeswax, carnauba wax, candelilla wax, ouricuri wax, apple wax, Japan wax, microcrystalline wax, ozocerite, synthetic wax, hydrated castor oil and the like, to achieve the desired purpose.

It was surprisingly found that in those basic materials with a creamy feel to the skin, it is also possible to have higher amounts of pigments—up to about 50% by weight and in combination with pearly luster pigments based on micas, even above that—, without detrimentally altering the desired product properties.

In principle the coloring agents referred to in the Cosmetic Regulations in Appendix 3 (in relation to §3) or those which are allowed by national laws in the USA or in Japan are suitable as pigments and can be used for cosmetics. By way of example and without limiting the use in respect thereof, mention may be made of the following here: titanium dioxide, iron oxides, ultramarine, chromium oxide green, chromium hydroxide green, Berlin blue, zinc oxide, mica, micas coated with metal oxides, bismuth oxychloride, metal powder such as for example flake-form aluminium, copper, bronze, brass, silver or gold, carmine, organic pigments, insoluble lakes of organic dyes or mixtures thereof. In addition it is possible to use further solids as fillers and consistency regulators such as for example talcum, kaolin, bentonite, hectorite, montmorillonite, smectite, magnesium aluminium silicate or metal soaps which are insoluble in water such as for example aluminium, magnesium, calcium or zinc stearate or mixtures of said solids.

The production of the cosmetic materials is effected by a procedure whereby the lipophilic components are brought together and at elevated temperature—about 10° C. above the melting temperature of the highest-melting component—caused to melt clear throughout and possibly filtered. Thereafter all solids such as pigments, fillers, anti-sedimentation agents and so forth and possibly preserving agents, anti-oxidants, fragrances, active substances and other additives are added and the whole is homogenised. It is also possible to add hydrophilic components such as for example water, glycerine, propylene-1,2-glycol, hexylene-1,2-glycol, pantothenol and also tocopheryl acetate or tocopherylinoleate or mixtures of said substances to the materials prior to homogenisation, to improve the cosmetic properties.

Then, to achieve a uniform particle size, crushing is effected with a corundum disk crusher or a bead crusher or a three-roller grinding unit. Thereafter air is removed from the material and the material is pressed to form leads with a suitable extruder. The leads are then glued into pieces of wood in the usual manner and processed to form cosmetic pencils.

It was found in accordance with the invention that it is precisely the combination of an ester as is defined in the claims and a silicone wax that provides that a cosmetic material which usually comprises a lipophilic phase with wax and oil becomes extrudable and, upon extrusion, such mechanical properties are achieved that it can be processed without any problem to form leads or refills and then pencils.

A further subject of the invention is therefore the use of a combination comprising a) an ester of a straight-chain or branched C₁-C₂₄ carboxylic acid and a straight-chain or branched C₁-C₂₄ alcohol and b) a silicone wax as an addition to a cosmetic preparation which has at least one lipophilic phase and which besides oils and waxes contains the usual ingredients in order to make the cosmetic preparation shapable by extrusion. The subject of the invention is also the use of said combination for the production of an extrudable cosmetic preparation.

The invention will be described hereinafter by reference to some examples without thereby limiting the invention. The quantitative units are in percent by weight (% by weight), and the raw materials are identified by the usual international INCI names.

### EXAMPLE 1

| | |
|---|---|
| Caprylic/capric triglycerides | 8.000 |
| Polyglyceryl-3 methylglucose distearate | 2.000 |
| Tri C 14-15 alkyl citrate | 5.000 |
| Hydrogenated palm glycerides-monoglyceride | 2.500 |
| Synthetic wax | 1.000 |
| Stearyl dimethicone | 20.000 |
| Trimethylsiloxysilicate | 7.500 |
| Dimethicone/vinyldimethicone crosspolymer | 2.500 |
| Pigments, mica, kaolin | 51.000 |
| Antioxidants | 0.500 |
| | 100.000 |

The coloring agents used are inorganic pigments and micas coated with titanium dioxide and optionally further metal oxides. The fat constituents are melted at 100° C., thereafter the pigments, fillers and silicone polymers are added and the material is rolled with a three-roller grinding unit and then extruded to form leads or refills for an eyeshadow pencil.

### EXAMPLE 2

| | |
|---|---|
| Caprylic/capric triglycerides | 2.500 |
| Polyglyceryl-3 methylglucose distearate | 1.500 |
| Tri C 14-15 alkyl citrate | 5.000 |
| Hydrogenated palm glycerides-monoglycerides | 3.000 |
| Synthetic wax | 1.500 |
| Stearyl dimethicone | 19.500 |
| Trimethylsiloxysilicate | 8.000 |
| Pigments, kaolin, mica | 52.500 |
| Aqua | 5.000 |
| Preservatives, antioxidants | 1.500 |
| | 100.000 |

The water (distilled water) is heated to 70° C. and the preserving agent system dissolved therein. The fat constituents are melted at 100° C. and allowed to cool to 80° C. The water phase is added and the mixture intensively mixed. Thereafter the pigments, fillers and silicone polymers are added. The mixture is then kneaded in a kneader for 30 minutes. Then the material is rolled on a three-roller rolling unit and extruded. Before the glueing operation the leads are dried at 50° C. in vacuum (40-50 kPa). When using inorganic pigments and micas coated with titanium dioxide and optionally further metal oxides, it is possible to produce leads for eyeshadow pencils. When using iron oxides mixed with titanium dioxide the result obtained is leads for correcting pencils and blusher.

### EXAMPLE 3

| | |
|---|---|
| Beeswax | 3.500 |
| Ouricuri wax | 2.500 |
| Isostearyl isostearate | 5.000 |
| Polyglyceryl-3 methylglucose distearate | 1.000 |
| Hydrogenated palm glycerides-monoglycerides | 3.000 |
| Stearyl dimethicone | 10.000 |
| Sucrose tetrastearate triacetate | 8.000 |
| Sucrose laurate | 1.000 |
| Trimethylsiloxysilicate | 7.500 |
| Pigments, kaolin, mica | 49.000 |
| Aqua | 3.500 |
| D-pantothenol | 2.500 |
| Hexylene-1,2-glycol | 2.000 |
| Preservatives, antioxidants | 1.500 |
| | 100.000 |

Production is effected in a similar manner to Example 2. The material is suitable for lipsticks with skin-care properties. If about 15-20% by weight of the specified amount of pigment is replaced by finely divided titanium dioxide with particle sizes around 20-50 nm, it is possible to produce lipsticks with a high light protection factor or—when selecting suitable inorganic pigments or organic pigments or color lacquer or mixtures thereof—pencils for body painting with a high light protection factor, as are popular for example with surfers or skiers. It is also possible by selecting suitable pigments to produce pencils of skin-tone shades which are suitable for the purposes of camouflage, for covering up skin anomalies.

1. A cosmetic preparation in the form of a lead containing a lipophilic phase comprising oils fluid at 25° C., waxes and a solid phase, wherein the lipophilic phase contains a combination of:
a) an ester selected from esters of citric acid with cetyl alcohol, esters of citric acid with stearyl alcohol, monoglycerides of palmitic or stearic acid or esters which are derived from methylglucose and stearic acid, saccharose esters with C₁₂-C₂₀ fatty acids, saccharose tetrastearate triacetate, polyglycerine-3-methylglucose distearate, jojoba oil and/or isostearyl isostearate or mixtures thereof and
b) a silicone wax selected from stearyl dimethyicone, polymer siloxysilicates of the general formula [CH₃)₂SiO_{1/2}]ₓ[SiO₂]_{y} or silicone elastomers of the type dimethicone/vinyldimethicone crosspolymer or mixtures thereof,
wherein the preparation is free of solid triglycerides with a melting range of between 30 and 60° C.

2. A cosmetic preparation as set forth in claim 1 characterised in that the lipophilic phase contains as component (a) tri-(cetyl/stearyl)-citrate and/or methylglucose distearate and/or palmitoyl/stearoyl monoglyceride and/or jojoba oil and/or isostearyl isostearate.

3. A cosmetic preparation as set forth in one of the preceding claims characterised in that it contains as component a) esters of citric acid with cetyl and stearyl alcohol, monoglycerides of palmitic and stearic acid or esters which are derived from methylglucose and stearic acid or mixtures thereof.

4. A cosmetic preparation as set forth in one of the preceding claims characterised in that it contains sucrose laurate, sucrose myristate, sucrose palmitate, sucrose stearate, sucrose tetrastearate triacetate or mixtures thereof.

5. A cosmetic preparation as set forth in one of the preceding claims characterised in that it contains as the solid phase, being admitted for cosmetics, coloring agents, pearly luster pigments based on mica, leaf-form metal powder, talcum, kaolin, bentonite, hectonite, montmorillonite, smectite, magnesium aluminium silicate, metal salts insoluble in water, or mixtures thereof.

6. A cosmetic preparation as set forth in one of the preceding claims characterised in that it contains aluminum stearate, magnesium stearate, calcium stearate, zinc stearate or mixtures thereof.

7. A cosmetic preparation as set forth in one of the preceding claims characterised in that it contains up to about 50% by weight of pigments.

8. A cosmetic preparation as set forth in one of the preceding claims characterised in that the lead was obtained by extrusion of the cosmetic preparation.

9. Use of a combination comprising
a) an ester of a straight-chain or branched C₁-C₂₄ carboxylic acid and a straight-chain or branched C₁-C₂₄ alcohol and
b) a silicone wax
as an addition to a cosmetic preparation which has at least one lipophilic phase and which besides oils and waxes contains the usual ingredients in order to make the cosmetic preparation shapable by extrusion.

10. Use of a combination comprising
a) an ester of a straight-chain or branched C₁-C₂₄ carboxylic acid and a straight-chain or branched C₁-C₂₄ alcohol and
b) a silicone wax
for the production of an extrudable cosmetic preparation.

11. Use as set forth in one of claims 9 and 10 for the production of leads with a pigment proportion of up to 50% by weight.

12. Use as set forth in one of claims 9 to 11 characterised in that component a) and/or component b) is as defined in one of claims 1 to 8.
